(19) United States
(12) Patent Application Publication
(10) Pub. No.: US 2010/0292664 A1
(43) Pub. Date: Nov. 18, 2010

(54) GARMENT HAVING A FLUID DRAINAGE LAYER

(75) Inventor: Robert Anthony Marin, Midlothian, VA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/776,596

(22) Filed: May 10, 2010

Related U.S. Application Data
(60) Provisional application No. 61/177,762, filed on May 13, 2009.

(51) Int. Cl. A61F 13/53; A41D 13/00; A41D 19/00; A42B 1/00

(52) U.S. Cl. 604/372; 2/69; 2/167; 2/200.1

(57) ABSTRACT

The present invention relates to a garment comprising a composite fabric comprising a porous fluid drainage layer and a fibrous fluid barrier layer of a flash spun polyolefin plexifilamentary film-fibril sheet wherein the fibrous fluid barrier layer faces the interior of the garment.

### FIELD OF THE INVENTION

The present invention relates to garments that contain a composite fabric having a porous fluid drainage layer and a fibrous fluid barrier layer, and optional outer and/or inner fabric layers enveloping the composite fabric.

### BACKGROUND

It has long been desired to make garments less susceptible to the strikethrough of fluids, e.g., water, blood and oil, through the garments, particularly at points or regions of high pressure, such as at elbows and the seat area of pants.

It is well known to incorporate breathable and waterproof membranes into a garment in order to impart breathability to the garment while making it waterproof and therefore more comfortable to the user. For example expanded polytetrafluoroethylene membranes (ePTFE) have been used for this application.

Over time, soil impingement in the form of body oils and exudates can severely affect membrane performance by blocking the pores in the membrane. Attempts to protect the membrane by coating it with polymers that allow passage of moisture vapor cause a reduction of the breathability of the garment. There is therefore a tradeoff between lifetime and comfort.

It would be desirable to provide comfortable garments with improved fluid strikethrough resistance, particularly at high-pressure areas.

### SUMMARY OF THE INVENTION

The present invention relates to a garment comprising a composite fabric comprising a porous fluid drainage layer and a fibrous fluid barrier layer of a flash spun polyolefin plexifilamentary film-fibril sheet wherein the fibrous fluid barrier layer faces the interior of the garment.

### DETAILED DESCRIPTION OF THE INVENTION

The term “garment” refers to any item that is worn by the user to cover or protect some region of the user's body from weather or other factors in the environment outside the body. For example coats, jackets, pants, hats, gloves, shoes, socks, and shirts are all considered garments under this definition.

The term “fluid drainage” layer refers to a layer wherein fluid can easily travel throughout the plane of the layer and can travel through the layer. By “through the layer” is meant that the fluid enters into one side of the layer and exits from the other side of the layer. The fluid has less resistance to flow throughout the plane of the fluid drainage layer than through the fluid barrier layer.

The term “fluid barrier” layer refers to a layer wherein fluid cannot easily travel through the layer. By “through the layer” is meant that the fluid enters into one side of the layer and exits from the other side of the layer. The fluid has more resistance to flow through the fluid barrier layer than throughout the plane of the fluid drainage layer.

The term “plexifilamentary film-fibril sheet” refers to a three-dimensional integral network of a multitude of thin, ribbon-like, film-fibril elements of random length and with a mean film thickness of less than about 4 microns and a median fibril width of less than about 25 microns. In plexifilamentary structures, the film-fibril elements are generally coextensively aligned with the longitudinal axis of the structure and they intermittently unite and separate at irregular intervals in various places throughout the length, width and thickness of the structure to form a continuous three-dimensional network. Traditionally, plexifilamentary film-fibril sheet is the spunbond sheet material that is produced by the process of flash spinning a polyolefin polymer solution.

The term “nonwoven” refers to a web including a multitude of randomly distributed fibers. The fibers generally can be bonded to each other or can be unbonded. The fibers can be staple fibers or continuous fibers. The fibers can comprise a single material or a multitude of materials, either as a combination of different fibers or as a combination of similar fibers each comprised of different materials.

The term “outer” when used to describe the location of a layer refers to the face of the garment that faces away from the wearer. The term “inner” refers to the user facing side of the garment.

The term “calendering” refers to the process of passing a web through a nip between two rolls. The rolls may be in contact with each other, or there may be a fixed or variable gap between the roll surfaces. An “unpatterned” roll is one which has a smooth surface within the capability of the process used to manufacture them. There are no points or patterns to deliberately produce a pattern on the web as it passed through the nip, unlike a point bonding roll.

The present invention relates to a garment comprising a composite fabric comprising a porous fluid drainage layer and a fibrous fluid barrier layer of a flash spun polyolefin plexifilamentary film-fibril sheet wherein the fibrous fluid barrier layer faces the interior of the garment. The fluid drainage layer restricts fluid strikethrough of fluid subjected to force or pressure, such as fluids being driven in by compression at elbows, seat area, etc., through the fluid barrier layer by reducing the pressure of the fluid which penetrates the fluid drainage layer, such that the fluid barrier layer is subjected to a significantly reduced fluid pressure. The fluid drainage layer is a flexible structure having a thickness, mean flow pore size and stiffness or crush resistance sufficient to allow fluid to quickly pass throughout the plane of the fluid drainage layer and away from the compressed region. In addition, it should be noted that the composite fabric can have a fluid drainage layer on both sides of the fluid barrier layer.

The fluid drainage layer can be selected from the group consisting of nonwoven fabrics, woven fabrics, knit fabrics, three-dimensional meshes, and combinations thereof. The nonwoven fabrics can be selected from the group consisting of spunbonded nonwovens, carded nonwovens, needlepunched nonwovens, air-laid nonwovens, wet-laid nonwovens, spunlaced and flash spun nonwovens. A particularly useful type of nonwoven fabric of the invention is a spunbonded nonwoven. As used herein, the term “spunbonded nonwoven” refers to a nonwoven web formed of filaments which have been extruded, drawn, and deposited on a continuous collection surface. Bonding can be accomplished by any of several methods including point or pattern bonding, calendering or passing the spunbonded nonwoven through a saturated-steam chamber at an elevated pressure.

The fluid drainage layer can be made from polymeric materials that are not particularly limited and include melt spinnable thermoplastic polymers such as polyolefins, polyesters, polyamides, and combinations thereof. Preferred polyolefins include polyethylene and polypropylene. A preferred polyester includes polyethylene terephthalate. Preferred polyamides include nylon 6 and nylon 6,6.

The fluid drainage layer can further comprise antistatic agents, antimicrobial agents, processing additives, colorants and the like.

The material of the fluid drainage layer can be non-absorbent and porous so that in the event that fluid penetrates to the fluid drainage layer, the fluid is less likely to fully wet the fluid drainage layer and/or penetrate to the fluid barrier layer. Alternatively, the material of the fluid drainage layer can be absorbent and have a high wicking rate so that the fluid will be quickly transported away from the penetration site.

The fluid drainage layer is optionally textured, creped, embossed or calendered to direct and facilitate the passage of fluid throughout the plane of the fluid drainage layer and away from the compressed region.

The fluid drainage layer can be treated with a fluorine-containing compound to help resist the penetration of certain solvents.

The fluid barrier layer resists the flow of fluids completely through the layer to keep the fluids from passing through the garment and contacting the wearer.

The fluid barrier layer is a flash spun polyolefin plexifilamentary film-fibril sheet. A preferred polyolefin is polyethylene.

The fluid barrier layer can be textured, creped, embossed or calendered.

The fluid barrier layer can be treated with a fluorine-containing compound to help resist the penetration of certain solvents.

The fluid drainage layer and fluid barrier layer can be optionally bonded to each other. Examples of suitable bonding techniques include adhesive bonding, thermal bonding such as point bonding, and ultrasonic bonding, although any means for bonding known to one skilled in the art can be employed.

In the case of adhesive bonding, the adhesive used can be any of a variety of adhesives, including dispersions and synthetic latexes. Preferably, the adhesive should have similar or better chemical resistance and thermal resistance properties than those of the fibers used in the composite fabric. Possible adhesive systems include aqueous anionic dispersions of butadiene acrylonitrile copolymers, copolymers based on acrylic esters, vinyl and vinylidene chloride polymers and copolymers produced by emulsion polymerization, styrene-butadiene copolymers, and terpolymers of butadiene, styrene, and vinyl pyridine.

Different methods of adhesively bonding the fluid drainage layer to the fluid barrier layer can be used. For example, one layer can be first coated in the required areas with adhesive and then the other layer is placed in contact with the adhesive on the first layer. Ample heat and pressure are applied to cause the adhesive to flow into some of the pores of the layers. If the adhesive is cross-linkable, the adhesive cross-links due to the heat and results in a mechanical attachment of the fluid drainage layer to the fluid barrier layer. In a preferred embodiment, the fluid drainage layer and the fluid barrier layer are bonded using a suitable lamination technique, such as passing the materials through a nip at a temperature sufficient to melt the adhesive. One of the nip rolls can have a raised pattern on its surface in order to produce a point bonding pattern in the laminate.

In the composite fabric, the fluid drainage layer directs fluid under pressure throughout the plane of the fluid drainage layer and resists the flow of fluid through the fluid barrier layer. General observations regarding the relative characteristics of the two layers can be described.

The mean flow pore size of the fluid drainage layer is preferably greater than the mean flow pore size of the fluid barrier layer allowing fluid to flow more readily through the fluid drainage layer than the fluid barrier layer. The mean flow pore size of the fluid drainage layer is preferably about 1 to about 6,000 microns and the mean flow pore size of the fluid barrier layer is preferably about 0.2 to about 50 microns.

The thickness of the fluid barrier layer needs to be at least thick enough to provide adequate resistance to fluid flow. However, the thickness of the fluid drainage layer can be any thickness provided that the fluid can flow throughout the plane of the fluid drainage layer. Generally, the thicker the fluid drainage layer is, then the greater the amount of fluid that can be transported away. The thickness of the fluid drainage layer is preferably greater than the thickness of the fluid barrier layer. The thickness of the fluid drainage layer is preferably about 0.08 to about 4 mm and the thickness of the fluid barrier layer is preferably about 0.08 to about 2 mm.

The stiffness or crush resistance of a layer is a measure of how intact the layer remains after pressure is applied. For the fluid drainage layer, the more crush resistant the layer is, then the more open or porous the layer remains and the more fluid that can be transported.

Stiffness or crush resistance can be affected by careful selection of fiber diameter. Generally, the larger the fiber diameter is, then the greater the layer crush resistance. Since it is desirable to maintain good crush resistance in the fluid drainage layer, then the use of large diameter fibers is preferred. Typically, larger diameter fibers are used in the fluid drainage layer than the fluid barrier layer. The fiber diameter of the fluid drainage layer is preferably about 3 to about 2,000 microns and the fiber diameter of the fluid barrier layer is preferably about 1 to about 40 microns.

In order for the fluid barrier layer to resist fluid flow through the fluid barrier layer, it is desirable for the fluid barrier layer to have a high hydrostatic head. Conversely, in order for the fluid drainage layer to be able to transport fluid away from the fluid barrier layer, the fluid drainage layer preferably has a lower hydrostatic head than the fluid barrier layer. The hydrostatic head of the fluid drainage layer is preferably about 3 to about 300 cm of water and the hydrostatic head of the fluid barrier layer is preferably about 3 to about 400 cm of water.

It is desirable for the garment of the present invention to be breathable for the comfort of the wearer. The Frazier air permeability for the garment may be about 0.1 to about 3,000 cm³/cm²/min, but for breathability is preferably about 300 to about 3,000 cm³/cm²/min.

The composite fabric is optionally located in the garment between an outer fabric layer and an inner fabric layer. A wide variety of natural and synthetic fabrics is known and may be used as the outer and inner fabric layers. Typically, vestments designed for use as rugged outerwear have been constructed of relatively loosely-woven fabrics made from natural and/or synthetic fibers having a relatively low strength or tenacity (for example, cotton, polyamides, polyesters, polyacrylics, polyolefins, and combinations thereof), with each fiber having a tensile strength or tenacity of less than about 8 grams per denier (gpd), more typically less than about 5 gpd, and in some cases below about 3 gpd. Such materials can have a variety of beneficial properties, for example, dyeability, breathability, lightness, comfort, and in some instances, abrasion-resistance.

Different weaving structures and different weaving densities may be used to provide several alternative woven fabrics as a component of the invention. Weaving structures such as plain woven structures, reinforced plain woven structures (with double or multiple warps and/or wefts), twill woven structures, reinforced twill woven structures (with double or multiple warps and/or wefts), satin woven structures, reinforced satin woven structures (with double or multiple warps and/or wefts) may be used. Stretch woven fabrics, ripstops, dobby weaves, and jacquard weaves are also suitable for use in the present invention.

Nonwoven fabrics can alternatively be used as the outer fabric layer and optional inner fabric layer. Examples of nonwoven sheets include spunbonded webs, melt blown webs, multi-directional, multi-layer carded webs, air-laid webs, wet-laid webs, spunlaced webs and composite webs comprising more than one nonwoven sheet.

The composite fabric may be bonded to the inner fabric layer and/or outer fabric layer over some fraction of its surface by any known means, for example adhesively, thermally, using an ultrasonic field, or by solvent bonding. One or more adhesives may optionally be used to bond the composite fabric to the inner and/or outer layer fabrics. One suitable adhesive is a thermoplastic adhesive, which can be softened upon heating, then hardened upon cooling over a number of heating and cooling cycles. An example of such a thermoplastic adhesive is a hot melt adhesive. In one embodiment the composite fabric is bonded adhesively using a solution of a polymeric adhesive such as a polyurethane, and allowing the solvent to evaporate.

In order to improve the liquid strikethrough resistance of the composite fabric and of the garment including the composite fabric, the outer fabric layer can be treated with known-in-the-art fluorine-containing compounds to improve the fluid repellency of the layer and render the outer fabric layer hydrophobic and oleophobic.

It can be advantageous to provide a hydrophilic treatment of the outer fabric layer in order to increase the wicking rate of the fluid drainage layer.

### TEST METHODS

Mean Flow Pore Size and Bubble Point were measured according to ASTM Designation E 1294-89 “Standard Test Method for Pore Size Characteristics of Membrane Filters Using Automated Liquid Porosimeter” which approximately measures pore size characteristics of membranes with a pore size diameter of 0.05 μm to 300 μm by using automated bubble point method from ASTM Designation F 316 using a capillary flow porosimeter (Porous Materials, Inc. (PMI), Ithaca, N.Y.). Individual samples (40 mm diameter) were wetted with low surface tension fluid “Sillwick,” having a surface tension of 20.1 dyne/cm, the sample is submerged in the fluid under a vacuum of 23 inches of mercury for a minimum of 20 minutes. Each sample was placed on a 16 mm diameter opening in a holder, and a differential pressure of air was applied and the fluid removed from the sample. The differential pressure at which wet flow is equal to one-half the dry flow (flow without wetting solvent) is used to calculate the mean flow pore size using supplied software. Bubble point refers to the biggest pore size.

Fiber Diameter was determined as follows. Ten scanning electron microscope (SEM) images at 5,000× magnification were taken of each fine fiber layer sample. The diameter of eleven (11) clearly distinguishable fine fibers were measured from the photographs and recorded. Defects were not included (i.e., lumps of fine fibers, polymer drops, intersections of fine fibers). The average (mean) fiber diameter for each sample was calculated.

Thickness was determined by ASTM D1777, which is hereby incorporated by reference, and is reported in mils and converted to microns.

Frazier Air Permeability is a measure of air permeability of porous materials and is reported in units of ft³/min per ft². It measures the volume of air flow through a material at a differential pressure of 0.5 inches water. An orifice is mounted in a vacuum system to restrict flow of air through sample to a measurable amount. The size of the orifice depends on the porosity of the material. Frazier permeability, which is also referred to as Frazier porosity, is measured using a Sherman W. Frazier Co. dual manometer with calibrated orifice units in ft³/ft²/min.

Hydrostatic Head is a measure of the resistance of the sheet to penetration by liquid water under a static load. A 7 inch×7 inch (17.78 cm×17.78 cm) sample is mounted in a SDL 18 Shirley Hydrostatic Head Tester (manufactured by Shirley Developments Limited, Stockport, England). Water is pumped against one side of a 102.6 cm² section of the sample at a rate of 60+/−3 cm/min until three areas of the sample are penetrated by the water. The hydrostatic pressure is measured in inches, converted to SI units and given in centimeters of water. The test generally follows ASTM D 583 (withdrawn from publication November, 1976).

What is claimed is:

1. A garment comprising a composite fabric comprising a porous fluid drainage layer and a fibrous fluid barrier layer of a flash spun polyolefin plexifilamentary film-fibril sheet wherein the fibrous fluid barrier layer faces the interior of the garment.

2. The garment of claim 1 wherein the fluid drainage layer is selected from the group consisting of nonwoven fabrics, woven fabrics, knit fabrics, three-dimensional meshes, and combinations thereof.

3. The garment of claim 2 wherein the nonwoven fabric is a spunbonded nonwoven.

4. The garment of claim 1 wherein the fluid drainage layer comprises fibers of a polymer selected from the group consisting of polyolefins, polyesters, polyamides, and combinations thereof.

5. The garment of claim 1 wherein the fluid drainage layer is non-absorbent.

6. The garment of claim 1 wherein the fluid drainage layer is absorbent.

7. The garment of claim 1 wherein the fluid drainage layer is textured, creped, embossed or calendered to facilitate the passage of fluid throughout the plane of the fluid drainage layer.

8. The garment of claim 1 wherein the fluid drainage layer is treated with a fluorine-containing compound.

9. The garment of claim 1 wherein the polyolefin is polyethylene.

10. The garment of claim 1 wherein the fluid barrier layer is textured, creped, embossed or calendered.

11. The garment of claim 1 wherein the fluid barrier layer is treated with a fluorine-containing compound.

12. The garment of claim 1 wherein the fluid drainage layer is bonded to the fibrous fluid barrier layer by means of adhesive bonding, thermal bonding, ultrasonic bonding, solvent bonding or any combination thereof.

13. The garment of claim 1 wherein the composite fabric is calendered.

14. The garment of claim 1 wherein the mean flow pore size of the fluid drainage layer is greater than the mean flow pore size of the fibrous fluid barrier layer.

15. The garment of claim 1 further comprising an outer fabric layer disposed adjacent to the fluid drainage layer opposite the fluid barrier layer and/or an inner fabric layer disposed adjacent to the fluid barrier layer opposite the fluid drainage layer.

16. The garment of claim 15 wherein the outer and inner fabric layers are nonwoven fabrics.

17. The garment of claim 15 wherein the outer and inner fabric layers comprise fibers of a polymer selected from the group consisting of cotton, polyamides, polyesters, polyacrylics, polyolefins, and combinations thereof.

18. The garment of claim 15 wherein the outer and inner fabric layers are bonded to the composite fabric by means of adhesive bonding, thermal bonding, ultrasonic bonding, solvent bonding or any combination thereof.

19. The garment of claim 15 wherein any layer is treated with a fluorine-containing compound.

20. A garment comprising a composite fabric comprising a porous fluid drainage layer of a spunbond nonwoven and a fibrous fluid barrier layer of a flash spun polyolefin plexifilamentary film-fibril sheet wherein the fibrous fluid barrier layer faces the interior of the garment.

21. The garment of claim 20 further comprising an outer fabric layer disposed adjacent to the fluid drainage layer opposite the fluid barrier layer and/or an inner fabric layer disposed adjacent to the fluid barrier layer opposite the fluid drainage layer.
